(19) United States
(12) Patent Application Publication
(10) Pub. No.: US 2021/0230531 A1
(43) Pub. Date: Jul. 29, 2021

(54) AUTOMATED AND DYNAMICALLY ADJUSTABLE GAS MIXER FOR BIOREACTOR SYSTEM

(71) Applicant: VBC Holdings LLC, Culver City, CA (US)

(72) Inventors: Giacomo CATTARUZZI, Fagagna (UD) (IT); Fabio TOMASINO, Tarcento (UD) (IT); Francesco ARMANI, Udine (UD) (IT)

(73) Assignee: VBC Holdings LLC, Culver City, CA (US)

(21) Appl. No.: 17/059,004

(22) PCT Filed: Jun. 5, 2019
(86) PCT No.: PCT/US2019/035483

§ 371 Date: Nov. 25, 2020

Related U.S. Application Data
(60) Provisional application No. 62/680,785, filed on Jun. 5, 2018.

(51) Int. Cl. C12M 1/34

(52) U.S. Cl. CPC C12M 41/34; C12M 41/40; C12M 41/12; C12M 41/26

(57) ABSTRACT

A gas mixer system includes a first line carrying a first gas component, and a first flow rate controller adapted to receive data relating to a measure of a first parameter and adjust a first flow rate of the first gas component based on the measure of the first parameter. The gas mixer system also includes a second line carrying a second gas component, and a second flow rate controller adapted to receive data relating to a measure of a second parameter and adjust a second flow rate of the second gas component based on the measure of the second parameter. The gas mixer system further includes a vessel connected to the first and second flow rate controllers, wherein the first gas component and the second gas component are mixed in the vessel to produce a gas mixture, and at least one outlet port. For example, the gas mixer may be connected to and provide a gas mixture to a bioreactor.

### FIELD OF THE INVENTION

The present invention relates generally to gas mixers and, and more particularly to automated and dynamically adjustable gas mixers for bioreactor systems.

### BACKGROUND OF THE INVENTION

There is a continuing need in the medical and biological sciences to conduct large numbers of experiments rapidly and effectively. For example, developing efficient and practical bioprocesses frequently requires testing a large number of different strains and environmental conditions in various combinations.

To meet this need, various types of bioreactors have been developed. A bioreactor typically provides an environment in which cells or tissues are grown in a cell culture. In many bioreactors, the cell culture is exposed to a gas mixture containing components such as oxygen and carbon dioxide in proportions selected to facilitate the growth of organisms in the cell culture.

Accordingly, an important component of many bioreactors is a gas mixer capable of providing a controlled flow of gas to the bioreactor environment. However, existing gas mixers are costly and do not provide a high degree of stability and reliability as a source of carbon dioxide, oxygen, and other gases. As a result, users of bioreactors often find it necessary to create custom-made solutions to provide the needed gas mixtures.

### SUMMARY OF THE INVENTION

In accordance with an embodiment, a bioreactor system is provided. The bioreactor system includes a bioreactor having a bioreactor environment comprising a volume adapted to hold one or more gases, a cell culture disposed in the bioreactor environment, and a sensor adapted to measure a parameter of the bioreactor environment. The bioreactor system also includes a gas mixer having a first line carrying a first gas component, a first flow rate controller adapted to adjust a first flow rate of the first gas component based on the parameter, a second line carrying a second gas component, a second flow rate controller adapted to adjust a second flow rate of the second gas component, a vessel connected to the first and second flow rate controllers, wherein the first gas component and the second gas component are mixed in the vessel to produce a gas mixture, and at least one outlet port through which the gas mixture is provided to the bioreactor environment.

In one embodiment, the first gas component includes one of: ambient air, oxygen, carbon dioxide, and nitrogen. The second gas component includes one of: ambient air, oxygen, carbon dioxide, and nitrogen.

In another embodiment, the parameter includes one of: a pressure of the bioreactor environment, a proportion of oxygen in the bioreactor environment, a proportion of carbon dioxide in the bioreactor environment, a proportion of nitrogen in the bioreactor environment, a measure of a growth rate of the cell culture, a pH level of the cell culture, and a temperature in the bioreactor environment.

In another embodiment, the sensor includes one of: a pressure sensor, an oxygen sensor, a carbon dioxide sensor, a nitrogen sensor, a growth rate sensor, a pH sensor, and a temperature sensor.

In another embodiment, the first gas component is carbon dioxide. The parameter is a pH level of the cell culture, and the first flow rate controller is adapted to adjust a flow rate of the carbon dioxide based on the pH level.

In another embodiment, the first gas component is oxygen. The parameter is a growth rate of the cell culture, and the first flow rate controller is adapted to adjust a flow rate of the oxygen based on the measure of the growth rate.

In another embodiment, the bioreactor system also includes a pressure regulator disposed between the vessel and the at least one outlet port.

In accordance with another embodiment, a gas mixer system is provided. The gas mixer system includes a first line carrying a first gas component, and a first flow rate controller adapted to receive data relating to a first parameter and adjust a first flow rate of the first gas component based on the first parameter. The gas mixer system also includes a second line carrying a second gas component, and a second flow rate controller adapted to receive data relating to a second parameter and adjust a second flow rate of the second gas component based on the second parameter. The gas mixer system also includes a vessel connected to the first and second flow rate controllers, wherein the first gas component and the second gas component are mixed in the vessel to produce a gas mixture, and at least one outlet port.

These and other advantages of the present disclosure will be apparent to those of ordinary skill in the art by reference to the following Detailed Description and the accompanying drawings.

### BRIEF DESCRIPTION OF THE DRAWINGS

FIG. 1 shows a schematic diagram of a bioreactor system in accordance with an embodiment;

FIG. 2 shows components of an automated gas mixer in accordance with an embodiment;

FIG. 3 shows functional components of a bioreactor in accordance with an embodiment;

FIG. 4A shows components of an air gas flow adjuster of a gas mixer in accordance with an embodiment;

FIG. 4B shows components of a CO₂ gas flow adjuster of a gas mixer in accordance with an embodiment;

FIG. 4C shows components of a gas mix precision pressure regulator of a gas mixer in accordance with an embodiment;

FIG. 5 is a flowchart of a method of controlling a gas mixture based on a pH level of a cell culture medium in accordance with an embodiment;

FIG. 6 is a flowchart of a method of controlling a gas mixture based on a growth rate of a cell culture in accordance with an embodiment; and

FIG. 7 shows an exemplary profile of oxygen levels within a gas mixture provided to a bioreactor environment in accordance with an embodiment.

### DETAILED DESCRIPTION

In accordance with an embodiment, a bioreactor system is provided. The bioreactor system includes a bioreactor having a bioreactor environment comprising a volume adapted to hold one or more gases, a cell culture disposed in the bioreactor environment, and a sensor adapted to measure a parameter of the bioreactor environment. The bioreactor system also includes a gas mixer having a first line carrying a first gas component, a first flow rate controller adapted to adjust a first flow rate of the first gas component based on the parameter, a second line carrying a second gas component, a second flow rate controller adapted to adjust a second flow rate of the second gas component, a vessel connected to the first and second flow rate controllers, wherein the first gas component and the second gas component are mixed in the vessel to produce a gas mixture, and at least one outlet port through which the gas mixture is provided to the bioreactor environment.

In one embodiment, the first gas component includes one of: ambient air, oxygen, carbon dioxide, and nitrogen. The second gas component includes one of: ambient air, oxygen, carbon dioxide, and nitrogen.

In another embodiment, the parameter includes one of: a pressure of the bioreactor environment, a proportion of oxygen in the bioreactor environment, a proportion of carbon dioxide in the bioreactor environment, a proportion of nitrogen in the bioreactor environment, a measure of a growth rate of the cell culture, a pH level of the cell culture, and a temperature in the bioreactor environment.

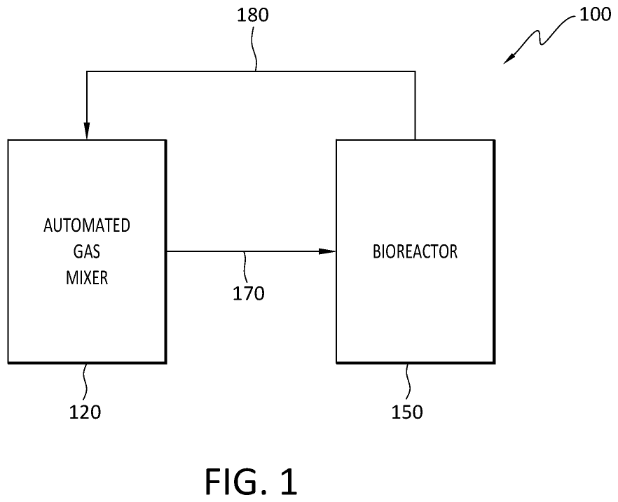

FIG. 1 shows a schematic diagram of a bioreactor system in accordance with an embodiment. The bioreactor system 100 includes a bioreactor 150 and an automated gas mixer 120. The bioreactor 150 holds a cell culture in a controlled environment. Gas mixer 120 provides a gas mixture 170 to bioreactor 150. The gas mixture 170 includes one or more gases (e.g., oxygen, carbon dioxide, nitrogen, ambient air, etc.) in specific proportions. Information 180 relating to various parameters of the bioreactor environment (e.g., amounts of certain gases, pH level, level of cell growth, etc.) are monitored and provided to the automated gas mixer 120. Gas mixer 120 continually adjusts one or more characteristics of the gas mixture 170 based on the information provided.

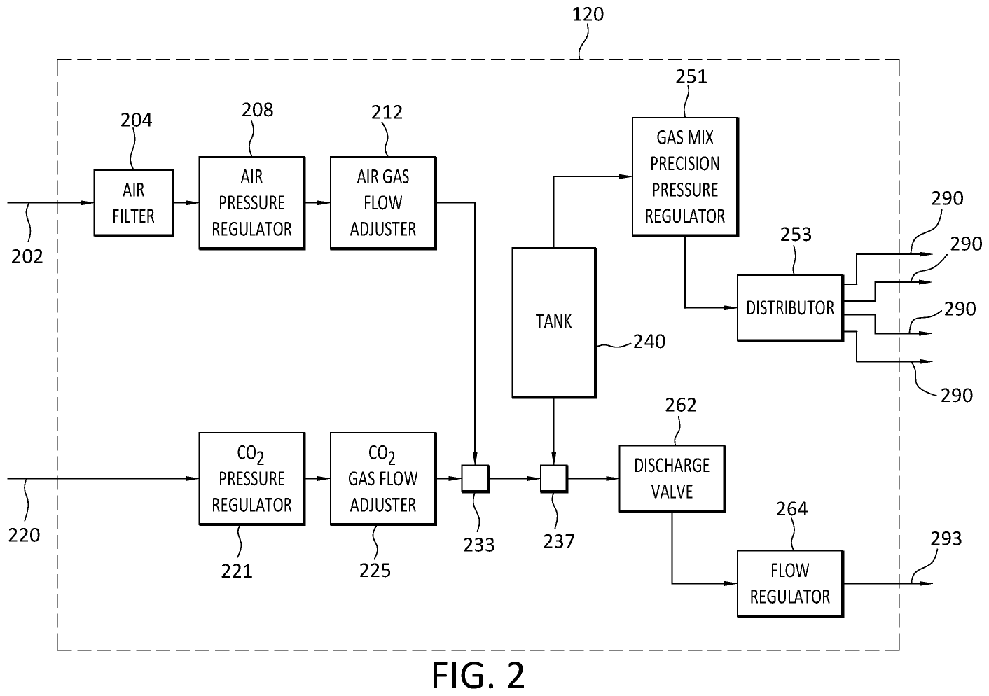

FIG. 2 shows components of automated gas mixer 120 in accordance with an embodiment. Gas mixer 120 includes an air filter 204, an air pressure regulator 208, an air gas flow adjuster 212, a carbon dioxide pressure regulator 221, a carbon dioxide gas flow adjuster 225, a tank 240, a gas mix precision pressure regulator 251, a distributor 253, a discharge valve 262, and a flow regulator 264. Automated gas mixer 120 also includes an air inlet port 202, a carbon dioxide inlet port 220, a discharge 293, and a plurality of carbon dioxide/air outlet ports 290. Gas mixer 120 also includes connectors 233 and 237 which direct the flow of gas between various components.

In accordance with an embodiment, gas mixer 120 is supplied with a 100% CO₂ line at 1.0 Bar (or higher) pressure. The pressurized CO₂ is supplied via inlet port 220. Ambient air is collected from the environment via inlet port 202 and compressed by a diaphragm pump. The ambient air is then filtered and dehumidified (e.g., by air filter 204). Air pressure regulator 208 and CO₂ pressure regulator 221 set pressure in the air line and pressure in the CO₂ line at 0.9 Bar. The filtered/dehumidified air and carbon dioxide are then supplied to air gas flow adjuster 212 and to CO₂ gas flow adjuster 225, respectively. Flow rate is set in order to obtain a 5% and a 95% over the total amount of 400 SCCM:20 SCCM CO₂ and 380 SCCM of Air.

Air and CO₂ are then mixed in tank 240, which may be, for example, a two-liter (2 L) pressure vessel. Discharge valve 262, which may be, for example, an overpressure actuated discharge valve, is connected to tank 240 and ensures that pressure of the gas mixture does not exceed 0.7 Bar. This serves as an overpressure safety control and as a means to keep the two flow regulators working in their linear control region.

Gas mix precision pressure regulator 251 is set to 0.5 Bar and connected to tank 240. Gas mix precision pressure regulator 251 provides a regulated 5% CO₂ gas mixture to outlet ports 290.

In accordance with an embodiment, automated gas mixer 120 provides to bioreactor 150 a gas mixture containing a plurality of components. Information relating to one or more parameters of the bioreactor environment are provided to the gas mixer, and the gas mixer dynamically adjusts the amounts of one or more of the components in the gas mixture based on the information.

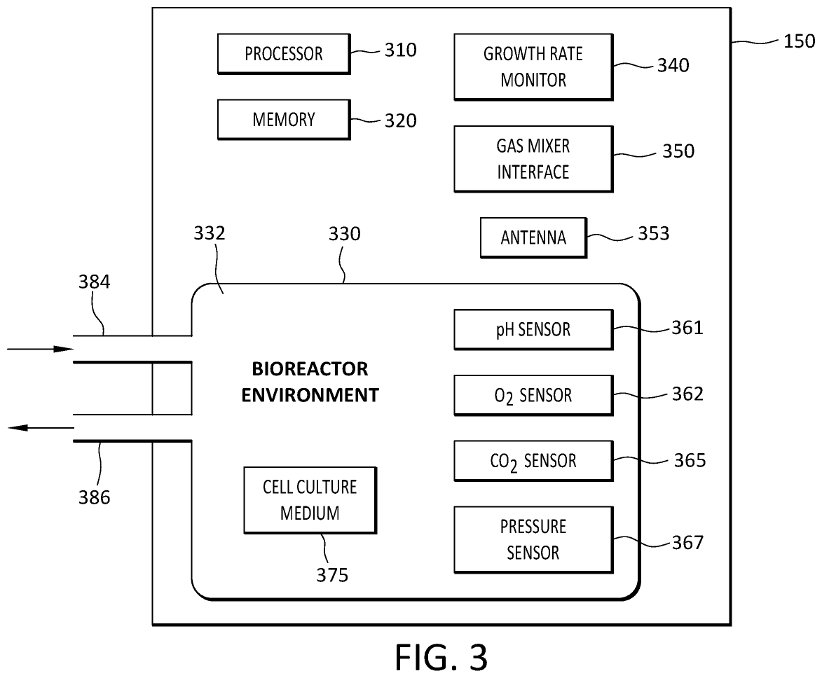

FIG. 3 shows functional components of bioreactor 150 in accordance with an embodiment. Bioreactor 150 includes a processor 310, a memory 320, a bioreactor environment 330, a growth rate monitor 340, and a gas mixer interface 350. Processor 310 orchestrates the operation of other components of bioreactor 150. Memory 320 stores data. For example, processor 310 or other components may from time to time store data in memory 320.

Bioreactor environment 330 is an enclosed volume adapted to hold a cell culture in a controlled environment. The enclosed volume can be any structure known in the field for growing cell cultures such as a cell culture bag, a cell culture flask or a cell culture tray. In the illustrative environment, a cell culture is maintained in a cell culture medium 375 within bioreactor environment 330. Bioreactor environment 330 also holds a gas mixture 332. Cell culture medium 375 is exposed to gas mixture 332. An inlet 386 allows gases to enter bioreactor environment 330. An outlet 384 allows gases to leave bioreactor environment 330.

Bioreactor environment 330 may include one or more sensors adapted to measure selected parameters of the environment. In the illustrative embodiment, a pH sensor 361, an O₂ sensor 363, a CO₂ sensor 365, and a pressure sensor 367 are disposed in bioreactor environment 330. The pH sensor 361 measures a pH level of cell culture medium 375. O₂ sensor 363 measures a level of oxygen within bioreactor environment 330. For example, O₂ sensor 363 may determine a proportion of oxygen in the air within bioreactor environment 330 (e.g., in the form of a percentage). CO₂ sensor 365 measures a level of carbon dioxide within bioreactor environment 330. For example, CO₂ sensor 363 may determine a proportion of carbon dioxide in the air within bioreactor environment 330 (e.g., in the form of a percentage). Pressure sensor 367 measures pressure within bioreactor environment 330.

In other embodiments, bioreactor 150 may include other types of sensors. For example, bioreactor 150 may include a temperature sensor, a scale adapted to measure a mass of the cell culture, etc.

Bioreactor 150 also includes a growth rate monitor 340 adapted to measure the growth rate of a cell culture in cell culture medium 375. Growth rate monitor 340 may include one or more cameras adapted to obtain images of the cell culture, and one or more sensors such as a temperature sensor, a scale adapted to measure the mass of the cell culture, etc. Growth rate monitor 340 analyzes images of the cell culture and measurements obtained by one or more sensors and determines a growth rate of the cell culture in cell culture medium 375. Growth rate monitor 340 may include an algorithm for estimating number of cells within a captured image, an algorithm for estimating area covered by cells within the captured image area, and an algorithm for calculating the increase of cells number and cells coverage in a certain amount of time.

Bioreactor 150 also includes a gas mixer interface 350. Gas mixer interface 350 transmits signals to automated gas mixer 120 based on information obtained by one or more sensors within bioreactor 150. In the illustrative embodiment, gas mixer interface 350 transmits information to automated gas mixer 120 via antenna 353.

In one embodiment, gas mixer interface 350 may transmit signals to various components of gas mixer 120. For example, gas mixer interface 350 may transmit information relating to one or more parameters (such as oxygen levels, carbon dioxide levels, pH levels, pressure, growth rates, etc.) to air gas flow adjuster 212, CO₂ gas flow adjuster 225, gas mix precision pressure regulator 251, and/or to other components of gas mixer 120.

In another embodiment, information relating to bioreactor 150 is provided to gas mixer 120 via electrical signals. For example, gas mixer interface 350 may transmit electrical signals to automated gas mixer 120 via one or more wires. The wires may link gas mixer interface to selected components of gas mixer 120. The wires may carry electrical signals containing information relating to measurements obtained by pH sensor 361, O₂ sensor 363, CO₂ sensor 365, pressure, the cell culture growth rate determined by growth rate monitor 340, and other parameters.

One or more components of automated gas mixer 120 receive information from bioreactor 150 and adjust the flow of selected gases, pressure levels of selected gases, and/or other operating conditions in response to the information. For example, certain components of gas mixer 120 receive information relating to oxygen levels, carbon dioxide levels, pH levels, pressure, growth levels, and/or other parameters, and adjust the flows of one or more gases based on these parameters.

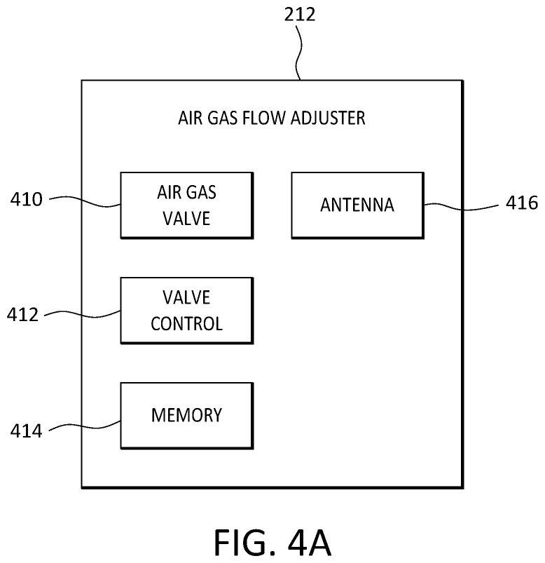

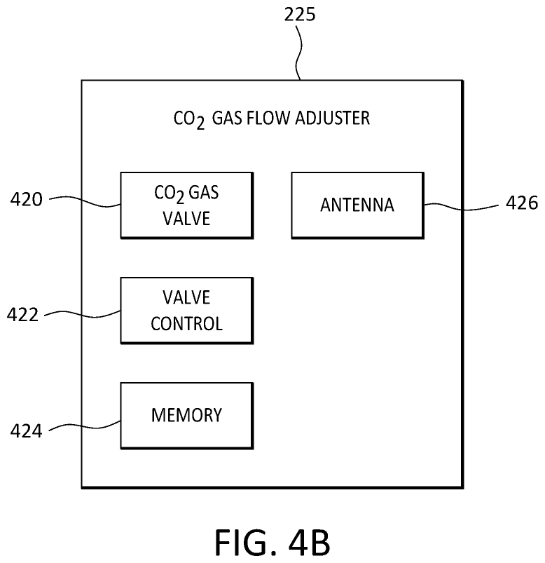

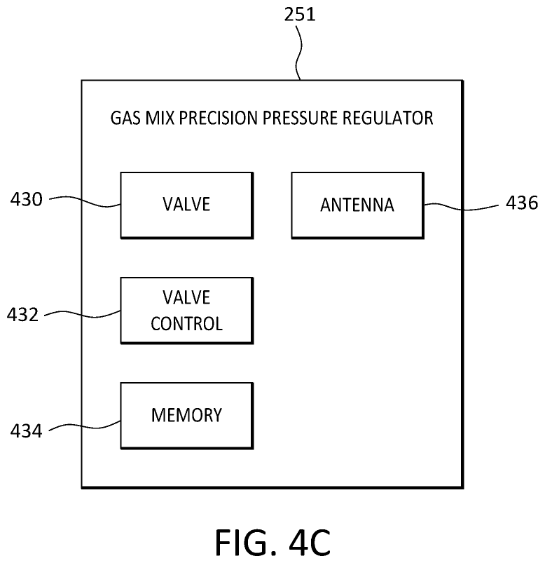

FIG. 4A shows components of air gas flow adjuster 212 of gas mixer 120 in accordance with an embodiment. Air gas flow adjuster 212 includes an air gas valve 410, a valve control 412, a memory 414, and an antenna 416. Antenna 416 receives signals from bioreactor 150 and transmits the signals to valve control 412. For example, the signals may relate to oxygen levels, carbon dioxide levels, pH levels, pressure, growth rate, and/or other parameters of the bioreactor environment. Valve control 412 controls air gas valve 410 in response to the signals receive by antenna 416. Signals received may be recorded in memory 414. Valve settings may also be recorded in memory 414.

FIG. 4B shows components of CO₂ gas flow adjuster 225 of gas mixer 120 in accordance with an embodiment. CO₂ gas flow adjuster 225 includes a CO₂ gas valve 420, a valve control 422, a memory 424, and an antenna 426. Antenna 426 receives signals from bioreactor 150 and transmits the signals to valve control 422. For example, the signals may relate to oxygen levels, carbon dioxide levels, pH levels, pressure, growth rate, and/or other parameters of the bioreactor environment. Valve control 422 controls CO₂ gas valve 420 in response to the signals receive by antenna 426. Signals received may be recorded in memory 424. Valve settings may also be recorded in memory 424.

FIG. 4C shows components of gas mix precision pressure regulator 251 of gas mixer 120 in accordance with an embodiment. Gas mix precision pressure regulator 251 includes a valve 430, a valve control 432, a memory 434, and an antenna 436. Antenna 436 receives signals from bioreactor 150 and transmits the signals to valve control 432. For example, the signals may relate to oxygen levels, carbon dioxide levels, pH levels, pressure, growth rate, and/or other parameters of the bioreactor environment. Valve control 432 controls valve 430 in response to the signals receive by antenna 436. Signals received may be recorded in memory 434. Valve settings may also be recorded in memory 434.

Gas mixer 120 provides a gas mixture that includes one or more gases to maintain a desired environment within bioreactor 150. For example, in one embodiment, gas mixer 120 may provide a gas mixture that includes two different gases. In another embodiment, gas mixer 120 may provide more than two gases (e.g., CO₂, N₂, O₂) in variable proportions, for example, to optimize cell growth.

Gas mixer 120 provides to bioreactor 150 a gas mixture that is appropriate for a cell culture maintained within bioreactor 150. For example, gas mixer 120 may provide a “standard” gas mixture containing 95% Air/5% CO₂, which is commonly used for many cell cultures, regardless of substrate-dependent growth or suspension growth. This is due to most cell culture media (CCM) being based on a carbonate buffer. In another embodiment, a 90% Air/10% CO₂ mixture may also be used in other carbonate-based buffered CCMs.

In accordance with an embodiment, various parameters and conditions associated with the environment within bioreactor 150 are monitored. The gas mixer is controlled based on one or more of these parameters and conditions.

Advantageously, the gas mixer 120 functions in a manner that is very simple, very precise, and robust compared to other commercial solutions that we tested.

It has been observed that because the environment within a bioreactor is dynamic, it can be challenging to maintain desired proportions of different gases within a bioreactor environment. Advantageously, the inventive bioreactor system monitors selected parameters within the bioreactor environment and dynamically adjusts the proportions of components in the gas mixture provided to the bioreactor environment based on the measured parameters, in order to maintain one or more desired conditions within the bioreactor environment.

In accordance with an embodiment, active controls are implemented in order allow the gas mixer to be adaptive with respect to the environment inside the bioreactor where cells are growing. Gas mixer 120 may adjust the proportion of a selected component of a gas mixture based on measurements of one or more parameters of the bioreactor environment.

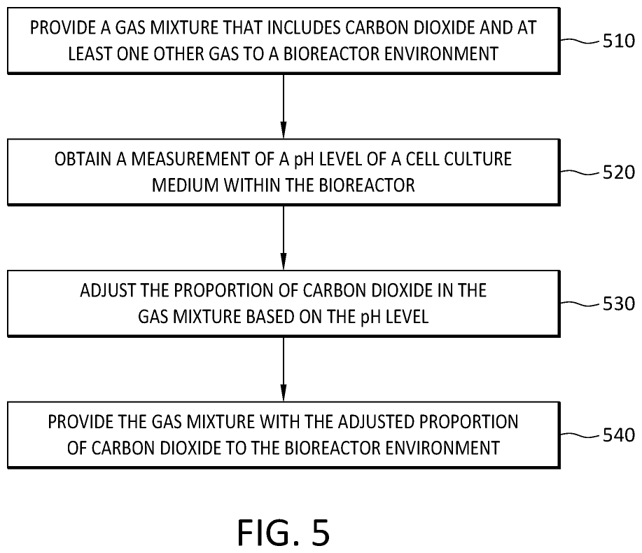

For example, in one embodiment, components of the gas mixture provided to bioreactor 150 are dynamically adjusted based on a measure of the pH level within the cell culture medium maintained in bioreactor 150. FIG. 5 is a flowchart of a method of controlling a gas mixture provided to a bioreactor environment based on a pH level of a cell culture medium in accordance with an embodiment.

At step 510, a gas mixture that includes carbon dioxide and at least one other gas is provided to a bioreactor environment. Gas mixer 120 provides a gas mixture to bioreactor 150. The gas mixture is provided to bioreactor environment 330 via inlet 386.

While the gas mixture is provided to bioreactor 150, the pH level of the cell culture medium within the bioreactor environment is monitored. For example, pH level within the cell culture medium may be measured by pH sensor 361. In one embodiment, pH sensor includes a contactless pH estimation subsystem.

At step 520, a measurement of a pH level of a cell culture medium within the bioreactor is obtained. In the illustrative embodiment of FIG. 3, pH sensor 361 measures the pH level of cell culture medium 375. The pH level measurement data is transmitted to gas mixer 120 via antenna 353.

Referring to FIG. 2, CO₂ gas flow adjuster 225 receives the pH level measurement data.

At step 530, the proportion of carbon dioxide in the gas mixture is adjusted based on the pH level. For example, when a decrease of pH is detected, the gas mixer may be controlled in order to alter (e.g., increase) the CO₂ percentage in the bioreactor. More specifically, if the pH level is below a predetermined level, the proportion of CO₂ within the gas mixture provided to the bioreactor is increased to a selected level. In the illustrative embodiment, CO₂ gas flow adjuster 225 may increase the proportion of carbon dioxide added to the gas mixture in response to the pH level measurement data.

At step 540, the gas mixture with the adjusted proportion of carbon dioxide is provided to the bioreactor environment. Gas mixer 120 provides the adjusted gas mixture to bioreactor 150. Referring to FIG. 3, the adjusted gas mixture is provided to bioreactor environment 330 via inlet 386.

In another embodiment, a level of carbon dioxide in the bioreactor environment (e.g., a percentage of CO₂ in the air within the bioreactor) is monitored. For example, this parameter may be measured by the CO₂ sensor 365.

In another embodiment, a level of oxygen in the bioreactor environment (e.g., a percentage of O₂ in the air within the bioreactor) is monitored. For example, this parameter may be measured by the O₂ sensor 363.

In accordance with an embodiment, the growth rate of the cell culture is monitored. This may be calculated, for example, using an integrated imaging system and confluency estimation SW, and, given the desired timing in which a certain confluency percentage is to be reached (es. x % in y days), may influence gas mixture composition. Imaging system may correspond to a lens or a group of lenses providing appropriate magnification to visualize cells, a light source to provide appropriate illumination of the area to be photographed, and a sensor to acquire photographs. Photographs are elaborated by a SW which provides an estimation of number of cells contained within the area of the acquired image, or an estimation of cell confluency (that is, percentage of area covered by cells over the total area of the acquired image). Growth rate is provided by calculating the increase of cells number or cells confluency in a certain amount of time.

In one embodiment, components of the gas mixture provided to bioreactor 150 are dynamically adjusted based on a measure of the growth rate of a cell culture within the cell culture medium maintained in bioreactor 150.

For example, if cell growth is determined to be below a predetermined level, oxygen level may be altered (e.g., decreased) to promote cell growth. It is well known that low oxygen concentrations (3-5%) enhance growth of mesenchymal stem cells (and other primary cells). Therefore, in one embodiment, in order to achieve a low oxygen concentration, CO₂, O₂ and N₂ (and no air) may be mixed together. In another embodiment, a mixture of air and carbon dioxide may be adjusted by decreasing the proportion of air in the mixture in order to achieve low oxygen levels.

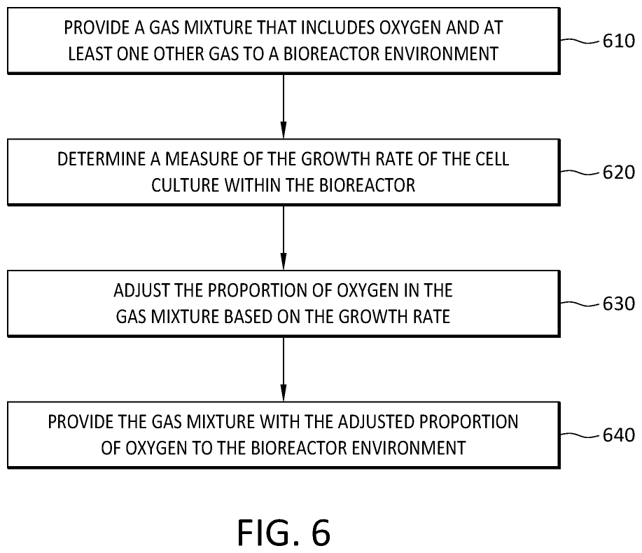

FIG. 6 is a flowchart of a method of controlling a gas mixture provided to a bioreactor environment based on a growth rate of a cell culture in accordance with an embodiment.

At step 610, a gas mixture that includes oxygen and at least one other gas is provided to a bioreactor environment. Gas mixer 120 provides a gas mixture to bioreactor 150. The gas mixture is provided to bioreactor environment 330 via inlet 386.

While the gas mixture is provided to bioreactor 150, the growth rate of the cell culture in cell culture medium 375 within bioreactor environment 330 is monitored by growth rate monitor 340.

At step 620, a measure of the growth rate of the cell culture within the bioreactor is obtained. In the illustrative embodiment of FIG. 3, growth rate monitor 340 determines the growth rate of the cell culture in cell culture medium 375. The growth rate data is transmitted to gas mixer 120 via antenna 353. Referring to FIG. 2, air gas flow adjuster 212 receives the growth rate data.

In the illustrative embodiment, suppose, for example, that the measured growth rate of the cell culture is (or falls) below a predetermined, desired level.

At step 630, the proportion of oxygen in the gas mixture is adjusted based on the growth rate level. For example, if the growth rate is determined to be below a predetermined level, the proportion of air within the gas mixture provided to the bioreactor may be decreased to a selected level (e.g., from an atmospheric level of 21% to as low as 5%). The decrease in the proportion of ambient air will in turn decrease the proportion of oxygen within the gas mixture if the other gas flow parameters remain unchanged. As known to those skilled in the art, cells cultured in low oxygen environment (i.e., subjected to hypoxia) will typically grow faster, live longer, and show lower stress. In the illustrative embodiment, air gas flow adjuster 212 may decrease the proportion of ambient air added to the gas mixture in response to the growth rate data.

At step 640, the gas mixture with the adjusted proportion of oxygen is provided to the bioreactor environment. Gas mixer 120 provides the adjusted gas mixture (with decreased oxygen levels) to bioreactor 150. Referring to FIG. 3, the adjusted gas mixture enters bioreactor environment 330 via inlet 386.

In accordance with an embodiment, the settings on gas mixer 120 related to various parameters, including percentages of selected gases (e.g., CO₂, O₂, N₂), pressure, and flow rates, are continuously recorded and used to provide a profile (plot) of the gas mixture administered to the bioreactor over time.

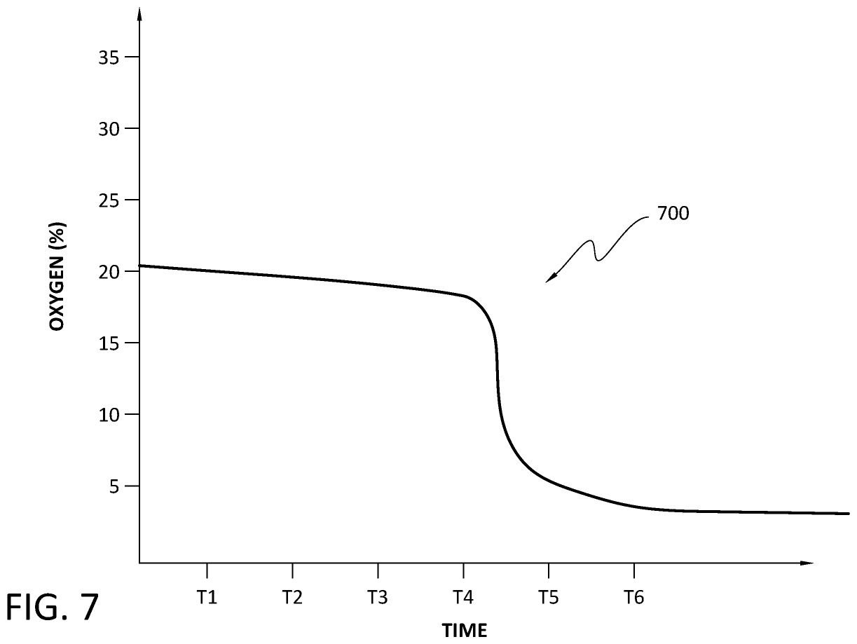

FIG. 7 shows an exemplary profile of oxygen levels within a gas mixture provided to a bioreactor environment 330 in accordance with an embodiment. In the illustrative embodiment, graph 700 indicates that oxygen levels fluctuated between 3% and 21% over a selected period of time. Profiles of other parameters may be generated.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A bioreactor system including:
a bioreactor comprising:
a bioreactor environment comprising a volume adapted to hold one or more gases;
a cell culture disposed in the bioreactor environment; and
a sensor adapted to generate a measure of a parameter of the bioreactor environment; and
a gas mixer comprising:
a first line carrying a first gas component;
a first flow rate controller adapted to adjust a first flow rate of the first gas component based on the measure of the parameter;
a second line carrying a second gas component;
a second flow rate controller adapted to adjust a second flow rate of the second gas component;
a vessel connected to the first and second flow rate controllers, wherein the first gas component and the second gas component are mixed in the vessel to produce a gas mixture; and
at least one outlet port through which the gas mixture is provided to the bioreactor environment.

2. The bioreactor system of claim 1, wherein:
the first gas component includes one of: ambient air, oxygen, carbon dioxide, and nitrogen; and
the second gas component includes one of: ambient air, oxygen, carbon dioxide, and nitrogen.

3. The bioreactor system of claim 1, wherein the parameter includes one of: a pressure of the bioreactor environment, a proportion of oxygen in the bioreactor environment, a proportion of carbon dioxide in the bioreactor environment, a proportion of nitrogen in the bioreactor environment, a measure of a growth rate of the cell culture, a pH level of the cell culture, and a temperature in the bioreactor environment.

4. The bioreactor system of claim 3, wherein the sensor comprises one of: a pressure sensor, an oxygen sensor, a carbon dioxide sensor, a nitrogen sensor, a growth rate sensor, a pH sensor, and a temperature sensor.

5. The bioreactor system of claim 1, wherein:
the first gas component is carbon dioxide;
the parameter is a pH level of the cell culture; and
the first flow rate controller is adapted to adjust a first flow rate of the carbon dioxide based on the pH level.

6. The bioreactor system of claim 1, wherein:
the first gas component is oxygen;
the parameter is a measure of a growth rate of the cell culture; and
the first flow rate controller is adapted to adjust a first flow rate of the oxygen based on the measure of the growth rate.

7. The bioreactor system of claim 1, further comprising:
a pressure regulator disposed between the vessel and the at least one outlet port.

8. A gas mixer system comprising:
a first line carrying a first gas component;
a first flow rate controller adapted to:
receive data relating to a measure of a first parameter; and
adjust a first flow rate of the first gas component based on the measure of the first parameter;
a second line carrying a second gas component;
a second flow rate controller adapted to:
receive data relating to a measure of a second parameter; and
adjust a second flow rate of the second gas component based on the measure of the second parameter;
a vessel connected to the first and second flow rate controllers, wherein the first gas component and the second gas component are mixed in the vessel to produce a gas mixture; and
at least one outlet port through which the gas mixture flows.

9. The gas mixer system of claim 8, wherein:
the first gas component includes one of: air, oxygen, carbon dioxide, and nitrogen; and
the second gas component includes one of: air, oxygen, carbon dioxide, and nitrogen.

10. The gas mixer system of claim 8, wherein:
the first parameter includes one of: a pressure of the bioreactor environment, a proportion of oxygen in the bioreactor environment, a proportion of carbon dioxide in the bioreactor environment, a proportion of nitrogen in the bioreactor environment, a measure of a growth rate of the cell culture, a pH level of the cell culture, and a temperature in the bioreactor environment; and
the second parameter includes one of: a pressure of the bioreactor environment, a proportion of oxygen in the bioreactor environment, a proportion of carbon dioxide in the bioreactor environment, a proportion of nitrogen in the bioreactor environment, a measure of a growth rate of the cell culture, a pH level of the cell culture, and a temperature in the bioreactor environment.
